(19) United States
(12) Patent Application Publication
(10) Pub. No.: US 2025/0040988 A1
(43) Pub. Date: Feb. 6, 2025

(54) MEDICAL COLLABORATIVE VOLUMETRIC ECOSYSTEM FOR INTERACTIVE 3D IMAGE ANALYSIS AND METHOD FOR THE APPLICATION OF THE SYSTEM

(71) Applicant: HOLOSPITAL KFT., Miskolc (HU)

(72) Inventors: Gergely DOBOS, Miskolc (HU); Zsolt MIHÁLYFI, Budaörs (HU); Richárd KISS, Budapest (HU); Olivér VASVÁRI, Budajeno (HU); James CHEN, Budapest (HU); Gergely HORVÁTH, Budapest (HU); Dorottya JUHÁSZ, Debrecen (HU); László BOGNÁR, Budakalász (HU); Péter NAGYIDAI, Budapest (HU); Márton PATAKI, Budapest (HU)

(73) Assignee: HOLOSPITAL KFT., Miskolc (HU)

(21) Appl. No.: 18/718,067

(22) PCT Filed: Dec. 8, 2021
(86) PCT No.: PCT/IB2021/061457

§ 371 Date: Jun. 10, 2024

(51) Int. Cl. A61B 34/10; A61B 34/20; G06T 15/08; G06T 19/00; G16H 30/20

(52) U.S. Cl. CPC A61B 34/10; G06T 15/08; G06T 19/003; G06T 19/006; G16H 30/20; A61B 2034/105; A61B 2034/2055; A61B 2034/2072; G06T 2210/41; G06T 2219/004; G06T 2219/024

(57) ABSTRACT

A medical collaboration system for pre-operative collaborative assessment includes an imaging center, a data center, a DICOM storage, at least one displaying means, an API and a rendering device. The data center includes a cloud storage, a user database, a DICOM converter and a web interface; the cloud storage includes a 3D medical volume storage; the imaging center is connected to the data center. The API is connected to the data center, the at least one displaying means, the rendering device and the 3D medical volume storage. The DICOM storage is connected to the DICOM converter and the DICOM storage is configured to send the 2D medical records to the DICOM converter. The DICOM converter is configured to remove confidential metadata from the 2D medical records, convert the 2D medical records into 3D medical volumes, and send the 3D medical volumes to the 3D medical volume storage for storing.

### CROSS REFERENCE TO THE RELATED APPLICATIONS

This application is the national phase entry of International Application No. PCT/IB2021/061457, filed on Dec. 8, 2021, the entire contents of which are incorporated herein by reference.

### TECHNICAL FIELD

The disclosure relates to a medical collaboration system for preoperative collaborative assessment and a method for the application of the medical collaboration system.

### BACKGROUND

During a preoperative assessment, medical imaging files and studies are the basis of medical consultations and analysis. In the current medical practice however, the storage, transfer and analysis of medical imaging studies is difficult for a number of reasons. The studies that are usually stored on a hospital's local area network, take up gigabytes/series of space on hard drives and thus their transfer is very problematic and slow. Furthermore, medical images are still distributed on DVDs by diagnostic companies. The sharing of the medical images requires high-bandwidth internet, gigabytes of data transfer per patient per study. Regarding image viewers, one of the problems is having to install a separate medical image viewer program on the computer or mobile device. Moreover, the image viewers are often too complicated, and their user interfaces can be confusing to doctors, because these software target radiologists. In general, a physician's workstation or personal device is not certified to store sensitive patient data, and does not have enough computational power for volumetric visualization. Another common issue is that medical cases require the consultation of specialists from several fields. This is often impossible because said professionals cannot make themselves available all at the same time with the necessary equipment to visualize the medical record and there are no specialized solutions for spatial communication in the virtual space.

The existing medical systems that support the analysis of medical records have several disadvantages. Some of them only visualize the structures in 2D, which is not sufficient for the thorough understanding of the anatomy of the patient. Even if 3D is used, many systems show the 3D medical data in such a way that computers and mobile devices are unable to process and visualize it. A further disadvantage of existing solutions is the lack of encryption of the medical studies, thus making sensitive patient data available to a high number of users.

Patent application No. US2013110537A1 discloses a cloud-based medical imaging viewer system and methods for non-diagnostic viewing of medical imaging. The system includes a cloud viewing network that interfaces with an electronic medical records system and provides a venue for secured consultations for authorized users. The system however does not use and analyze in 3D. This is a serious problem as most pathological structures can only be analyzed in 3D.

Patent No. U.S. Pat. No. 10,499,997B2 describes a system and a method for surgical navigation providing mixed reality visualization via a head-mounted display worn by the user. The registration device uses a plurality of markers (registration and tracking markers) during the process, which makes the method slow, cumbersome and inaccurate, since navigation probes must be placed at locations on the patient's bone. This requires a large amount of accurate and professional medical work before every surgery, making the method unnecessarily long and expensive. Using markers during the registration process can also be riskier for the patients, since—in most cases—it increases the time spent under anesthesia.

### SUMMARY

It is an object to provide an improved medical collaboration system. The foregoing and other objects are achieved by the features of the independent claims. Further implementation forms are apparent from the dependent claims, the description, and the figures.

According to a first aspect, there is provided a medical collaboration system for pre-operative collaborative assessment, including an imaging center, a data center, a Digital Imaging and Communications in Medicine (DICOM) storage, at least one displaying means having annotation tools, an application programming interface (API) and a rendering device; the data center including a cloud storage, a user database, a DICOM converter and a web interface; the cloud storage including a 3D medical volume storage; the imaging center being connected to the data center and the imaging center being configured to obtain 2D medical records from a Picture Archiving and Communication System (PACS) server and/or from a disk and/or from an imaging machine and send the 2D medical records to the DICOM storage; the API being connected to the data center, the at least one displaying means, the rendering device and the 3D medical volume storage; the DICOM storage being connected to the DICOM converter and the DICOM storage being configured to send the 2D medical records to the DICOM converter; the DICOM converter being configured to remove confidential metadata from the 2D medical records, convert the 2D medical records into 3D medical volumes, and send the 3D medical volumes to the 3D medical volume storage for storing; the user database including a list of authorised users; the rendering device being configured to render at least one 3D medical volume on the at least one displaying means in response to an input from an authorised user; the at least one displaying means being configured to display the at least one 3D medical volume, the API being configured to allow the authorised user to create a board, the board including at least one 3D medical volume; the API being configured to allow the authorised user set display parameters and annotate and/or comment on the at least one 3D medical volume in the board, the user database being configured to save and store the board with the annotations and/or comments and the associated display parameters; and the rendering device being configured to render the same board with the saved annotations and/or comments and the associated display parameters on the at least one displaying means in response to an input from the same or a different authorised user.

This solution provides a medical collaborative volumetric ecosystem for interactive 3D image analysis that helps increase quality assurance in healthcare. An advantage of the system is that it can open any 2D medical records (such as CT, MRI, Xray, Ultrasound, etc.) from any hospital around the globe once the necessary connection is established. In the system, users can view, rotate, scale and cut the at least one 3D medical volume in the board with a clipping plane from any angle. Users can also annotate on the at least one 3D medical volume in the board spatially in 3D by selecting the desired point on the surface or the inner part of the volume. Annotation can be done via text and/or voice input. The architecture of the backend has rapidly scalable cloud modules so the system can balance the load of millions of users coming from various continents using its cloud architecture on server farms across the globe.

In a possible implementation form of the first aspect, the system also includes a navigation arrangement for intra-operative use, the navigation arrangement being connected to the data center and including an XR (Extended Reality) device, a depth-camera, a tracking sensor, registration device and a navigation rendering device; the tracking sensor being connected to a surgical tool; the registration device being connected to the depth-camera and to the 3D medical volume storage the navigation rendering device being connected to the user database, to the XR device, to the tracking sensor and to the registration device; the registration device being configured to prepare a virtual image by registering at least one 3D medical volume onto a patient's anatomical structure; and the navigation rendering device being configured to render the virtual image received from the registration device with the saved annotations and/or comments received from the user database on the XR device in real time. This facilitates performing safe, fast and more precise operations, real-time optical navigation of the surgical tools and displaying the annotations and/or comments to a surgeon performing a surgery.

In a further possible implementation form of the first aspect, the XR device is a head-mounted XR display and at least one depth-camera is integrated in the XR device. This facilitates a convenient and safe solution in the intraoperative situation, for example for a surgeon doing an operation. The head-mounted XR display can be AR glasses, which enable the surgeon to receive wide range of navigational information while maintaining focus on the surgical site and/or surgical tools.

In a further possible implementation form of the first aspect, the rendering device is a remote rendering server. This facilitates remote rendering in real time. Remote volumetric rendering bypasses the hurdle of storing huge and sensitive data on client devices and displaying means that do not have enough computational power to visualize it. 3D medical volumes and/or boards are processed and rendered on a remote server, which provides physicians with an interactive 3D viewer and annotation tools for the 2D/3D records from a displaying means, for example the browser of any computer, mobile device, or vehicle. A remote rendering online approach can also allow the patients to examine their own studies via a simple link and forward it to another doctor for a second opinion.

In a further possible implementation form of the first aspect, the displaying means is any of a cell phone, a tablet, a computer and a web browser. This allows the usage of a broad range of devices for both viewing, annotating and commenting, providing convenience for all users, such as patients and doctors.

In a further possible implementation form of the first aspect, the DICOM storage is in the imaging center and/or in the cloud storage. This facilitates the flexible arrangement of the system, since the DICOM storage can be located at the hospital, in the cloud managed by the service provider or at both locations.

According to a second aspect, there is provided a method for the application of a medical collaboration system, the method including the steps of: an imaging center obtaining a 2D medical record from a Picture Archiving and Communication System (PACS) server and/or from a disk and/or from an imaging machine, the imaging center sending the 2D medical record to a Digital Imaging and Communications in Medicine (DICOM) storage; the DICOM storage sending the 2D medical record to a DICOM converter; the DICOM converter removing confidential metadata from the 2D medical record, converting the 2D medical record into a 3D medical volume, providing the 3D medical volume with a unique identification and sending the 3D medical volume to a 3D medical volume storage for storing; a user requesting access to the medical collaboration system via the API; a data center authorising the user by checking a user database in the data center; after authorisation, allowing the authorised user access; a rendering device rendering at least one 3D medical volume on at least one displaying means in response to an input from the authorised user; the at least one displaying means displaying the at least one 3D medical volume, the authorised user creating a board, the board including at least one 3D medical volume; the authorised user setting display parameters and annotating and/or commenting on the at least one 3D medical volume in the board, the user database saving and storing the board with the annotations and/or comments, with the 3D coordinates of the annotations and/or comments, and the associated display parameters; and the rendering device rendering the same board with the saved annotations and/or comments and the associated display parameters on the at least one displaying means in response to an input from the same or a different authorised user.

This solution provides a method allowing users to create boards for each medical case, which can contain different modalities or time-varying sequences-like pre/postoperative records for progression tracking. This collaborative board creates a virtual medical council where physicians can be remote. Specialists can work asynchronously when they view, spatial annotate, spatial comment on the volumetric datasets from any displaying means. Annotation and comments of experts can be summarized in video meetings by invited collaborators, where the common understanding of biological 3D structures makes communication more effective between different medical fields. Digital consultation is not only more practical, but it is the only solution if doctors and patients cannot physically meet each other. A ‘presentation mode’ can also be used, where the presenter user's point of view shared with the collaborators who joined the board. In this mode, viewers can get the position, rotation, clipping plane, and image properties e.g. threshold, look-up-table, brightness, contrast, etc. of the 3D medical volume. During the collaborative online case presentation, the viewers can see the 3D spatial pointer device of the presenter, therefore he or she can accurately show the 3D biological structures and its contexts for the sake of common understanding.

In a possible implementation form of the second aspect, the method further includes the steps of an authorised user choosing a board including at least one 3D medical volume;
a depth-camera sending a 3D point cloud of a patient's anatomical structure to a registration device; the 3D medical volume storage sending a 3D point cloud of the 3D medical volume to the registration device; the registration device registering the two 3D point clouds onto each other and creating a virtual image by doing a calculation including the steps of:
   pre-sampling vertices of the two 3D point clouds according to the Poisson distribution,
   calculating the normal vectors at each point,
   sampling a number of sub point clouds from the 3D point clouds,
   using a neural net to generate descriptive feature vectors,
   comparing these vectors by computing their Euclidean Distance and finding their best matching sub point clouds in the 3D point clouds, coming from the depth-camera,
   finding the most exactly matching sub point clouds, and using their corresponding Transformation Matrix on the two 3D point clouds.

This facilitates the substantial reduction of the time required for surgical preparations. This is a huge advantage, since—in order to decrease the risk of complications—the time a patient is kept under anesthesia should be as short as possible. Thus, the method can also be used in emergency patient care. This also makes it possible to do a surgical navigation without using physical markers and without needing human power during surgery preparations, making the method less expensive.

In a possible implementation form of the second aspect, the 3D point cloud coming from the 3D medical volume storage is registered onto the 3D point cloud coming from the depth-camera, and wherein the number of sub point clouds sampled from the two 3D point clouds coming from the depth-camera is lower than the number of sub point clouds sampled from the 3D point cloud coming from the 3D medical volume storage. This facilitates registering the preoperative point cloud (coming from the 3D medical volume storage) onto the depth-camera's point cloud.

In a possible implementation form of the second aspect, the method further includes the steps of the registration device sending the virtual image to a navigation rendering device; the user database sending the saved annotations and/or comments from the chosen board to the navigation rendering device; and the navigation rendering device rendering the virtual image with the saved annotations and/or comments on the XR device in real time. This enables doctors, such as surgeons to view their own or their colleagues' annotations and/or comments projected onto the patient's anatomical structures in real time, while performing an operation. This facilitates quicker and safer operations and real-time optical navigation of the surgical tools. The annotations and/or comments and/or the navigation are preferably displayed on the XR device using augmented reality. To make the method even safer, method can be done without internet, since the boards including the 3D medical volume with annotations and/or comments can be set to be available offline in the hospital intranet system.

In a possible implementation form of the second aspect, the method further includes a precomputation step before the depth-camera sends a 3D point cloud of a patient's anatomical structure to a registration device, the precomputation step including:
pre-sampling vertices of the 3D point cloud coming from the 3D medical volume storage according to the Poisson distribution,
calculating the normal vectors at each point,
sampling a number of sub point clouds from the 3D point cloud coming from the 3D medical volume storage,
using a neural net to generate descriptive feature vectors.

This facilitates an even quicker optical registration and navigation during intra-operative use. Quicker method results in safer operations.

In a possible implementation form of the second aspect, a new board is created for every medical case. This facilitates keeping a board for example for pre and postoperative records for progression tracking and creating a virtual medical council for each board to which physicians can join remotely. This enables the discussion of each medical case by specialists, who can join a video call or work asynchronously by creating annotations and comments on the board. Thus, this facilitates medical consultations by different professionals at the same or at a different time.

This and other aspects will be apparent from the embodiments described below.

### BRIEF DESCRIPTION OF THE DRAWINGS

In the following detailed portion of the present disclosure, the aspects, embodiments and implementations will be explained in more detail with reference to the example embodiments shown in the drawings, in which:

FIG. 1 shows a possible layout of the system in accordance with one embodiment of the present invention;

FIG. 2 shows a possible layout of the data center of the system in accordance with one embodiment of the present invention;

FIG. 3 shows another possible layout of the data center of the system in accordance with one embodiment of the present invention;

FIG. 4 shows a possible layout of a part of the system in accordance with one embodiment of the present invention;

FIG. 5 shows a possible layout of the navigation arrangement of the system in accordance with one embodiment of the present invention;

FIG. 6 shows a possible layout of a part of the system for intraoperative use in accordance with one embodiment of the present invention.

### DETAILED DESCRIPTION OF THE EMBODIMENTS

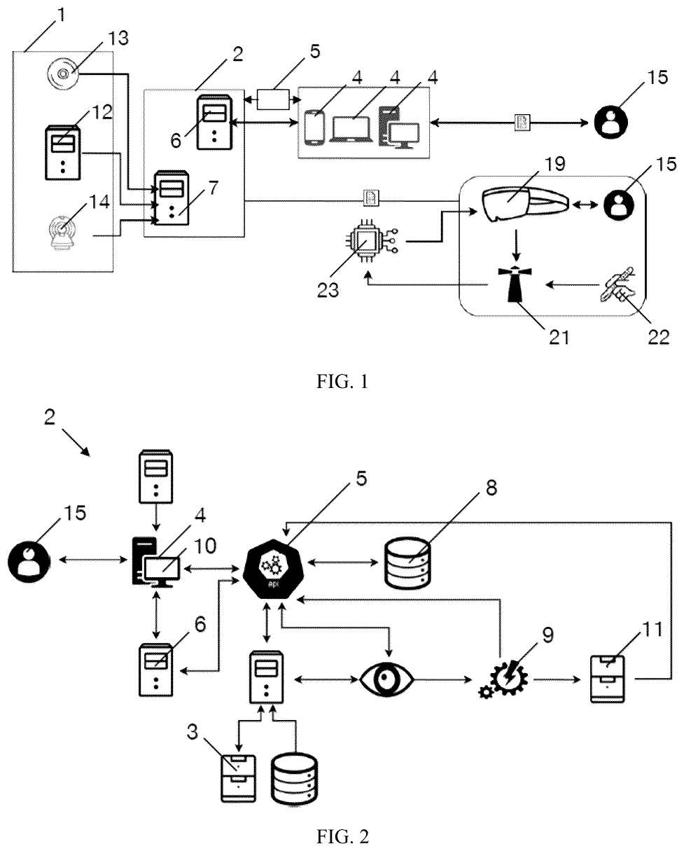

FIG. 1 illustrates a possible embodiment of the medical collaboration system for pre-operative collaborative assessment. The system preferably includes an imaging center 1, a data center 2, a Digital Imaging and Communications in Medicine (DICOM) storage 3, at least one displaying means 4, an application programming interface (API) 5 and a rendering device 6. The DICOM storage 3 may be in the imaging center 1 and/or in the cloud storage 7. This means that there might be more DICOM storages 3; there can be DICOM storages 3 in each hospital and/or the hospitals can use the system's cloud storage 7. The displaying means 4 may be any of a cell phone, a tablet, a computer and a web browser. The API 5 is a central part of the system. The annotations and/or comments 26 are sent to and controlled by the API 5. The identification and authorisation of the users is also done via the API 5. In order to increase safety, the authorisation of the users is preferably not automatic. The data center 2 preferably includes a cloud storage 7, a user database 8, a DICOM converter 9 and a web interface 10. The cloud storage 7 is preferably a HIPAA-compliant cloud storage with a database to guarantee fast and safe access from any device. The cloud storage 7 preferably includes a 3D medical volume storage 11. The imaging center 1 is preferably connected to the data center 2 and its task is obtaining 2D medical records from a Picture Archiving and Communication System (PACS) server 12 and/or from a disk 13 and/or from an imaging machine 14 and sending the 2D medical records to the DICOM storage 3. The 2D medical records are for example DICOM files. The 2D medical records are stored in the DICOM storage 3 in their original version, without any modifications or alterations. The PACS server, the disk(s) 13 and the imaging machine(s) 14 are not part of the invention. The disk 13 is for example CD or DVD. The imaging machine is for example a CT, MRI, X-ray or an Ultrasound machine. The API 5 is preferably connected to the data center 2, the at least one displaying means 4, the rendering device 6 and the 3D medical volume storage 11. The DICOM storage 3 is preferably connected to the DICOM converter 9 its task is sending the 2D medical records to the DICOM converter 9. The DICOM converter 9 removes confidential metadata, such as patient information from the 2D medical records, converts the 2D medical records into 3D medical volumes 25, and sends the 3D medical volumes 25 to the 3D medical volume storage 11 for storing. Preferably, each 3D medical volume 25 is provided with a unique identification code. The user database 8 includes a list of authorised users 15. The task of the rendering device 6 is rendering at least one 3D medical volume 25 on a displaying means 4 in response to an input from an authorised user 15. The input may be via the web interface 10, via a displaying means 4, via audio input, etc. The task of the at least one displaying means 4 is displaying the at least one 3D medical volume 25. The authorised user 15 can create a board 16 for each medical case, the board 16 includes at least one 3D medical volume 25, but it can include any number of 3D medical volumes 25. The authorised user(s) 15 can also set display parameters such as viewing angle, rotation and zoom, and make annotations and/or comments 26 on any of the 3D medical volumes 25 in the board 16. The authorised user(s) 15 can also view, rotate, transform, scale and clip the 3D medical volume 25 with a clipping plane from any angle. Web browser frontend sends the interactions-like slider values and button click events to the rendering device 6, where these events are decoded to change the properties of the rendition (colors, opacity, thresholds, etc.). The rendering device 6 may be a remote rendering server, or the rendering may take place locally on the users' 15 computers or displaying means 4, for example using a desktop client. This desktop client or desktop app is able to render the boards 16 locally for desktop use or holographic remoting. The 3D medical volumes 25 with the associated display parameters and with the annotations and/or comments 26 added by any authorised users 15 can be all saved and stored in the user database 8. The user database 8 thus stores all user related information, boards 16, settings, last opened boards 16 by the authorised user 15 and search history. The authorised users 15 can comment on the annotations or start a chat under the 3D medical volumes 25. The authorised users 15 can also mention other users who will get a notification to react. Any number of boards 16 can be created. The boards 16 can contain different modalities or time-varying sequences-like pre/postoperative records for progression tracking. Each board 16 creates a virtual medical council to which physicians, doctors and other medical professionals can join remotely. Experts and specialists can discuss the case in a video call or work asynchronously by creating annotations and/or comments 26 on the board 16. The rendering device 6 can render the board(s) 16 with the saved annotations and/or comments 26 and the associated display parameters on the at least one displaying means 4 in response to an input from any different authorised user(s) 15. This way, the patients and/or the authorised users 15 can open the boards 16 at any time and will see the comments of the physicians, doctors and other medical professionals. This allows patients to examine their own cases, studies via a simple link and forward it to another doctor for a second opinion.

The authorised user(s) 15 who are currently viewing a board 16 are preferably listed on the displaying means 4. When an authorised user 15 is editing the board 16, the pointer preferably moves on the surface of the clipping plane and a mouse click, so that the authorised user 15 can place the annotation on the selected surface. In the case of segmented, thresholded three-dimensional content—like angiography, tractography, or segmented pathologies—the pointer can move on the surface of the spatial structure. Spatial annotations and comments are in the same coordinate system across registered volumes. Besides the written information and the 3D coordinates, the annotation contains all the visualization settings of the creator when it was made to make coming back to them unambiguous (i.e. the volume is rendered exactly the same way). All experts (physicians, doctors and other medical professionals, etc.) involved in the consultation can place spatial annotations or leave a comment on the existing ones. The comments are signed with the ID of the authorised user's 15 profile for quality assurance. The board(s) 16 can be set to be available offline in the system. Experts' annotations and/or comments 26 can be summarized in video meetings by invited collaborators, where the common understanding of biological 3D structures makes communication more effective between the different fields. Digital consultation is not only more practical, but it is the only solution if doctors and patients cannot physically meet each other.

The board(s) 16 can be viewed in a ‘presentation mode’, when the presenter's point of view is shared with the collaborators who joined the board. Both the presenter and the collaborators are authorised users 15. The viewers get the position, rotation, clipping plane, and image properties e.g. threshold, look-up-table, brightness, contrast, etc. of the 3D medical volumes 25. During the collaborative online case presentation, the viewers can see the 3D spatial pointer device of the presenter, therefore he or she can accurately show the 3D biological structures and its contexts for the sake of common understanding. When an agreement is reached between specialists, each user signs the board (the summary of data, annotations, and comments) with his or her digital signature. After a board 16 is signed, it is considered finished and it can be modified further without first invalidating all signatures.

The authorised users 15 may be human or non-human, including persons, machines, devices, neural networks, robots and algorithms, as well as heterogeneous networked teams of persons, machines, devices, neural networks, robots and algorithms.

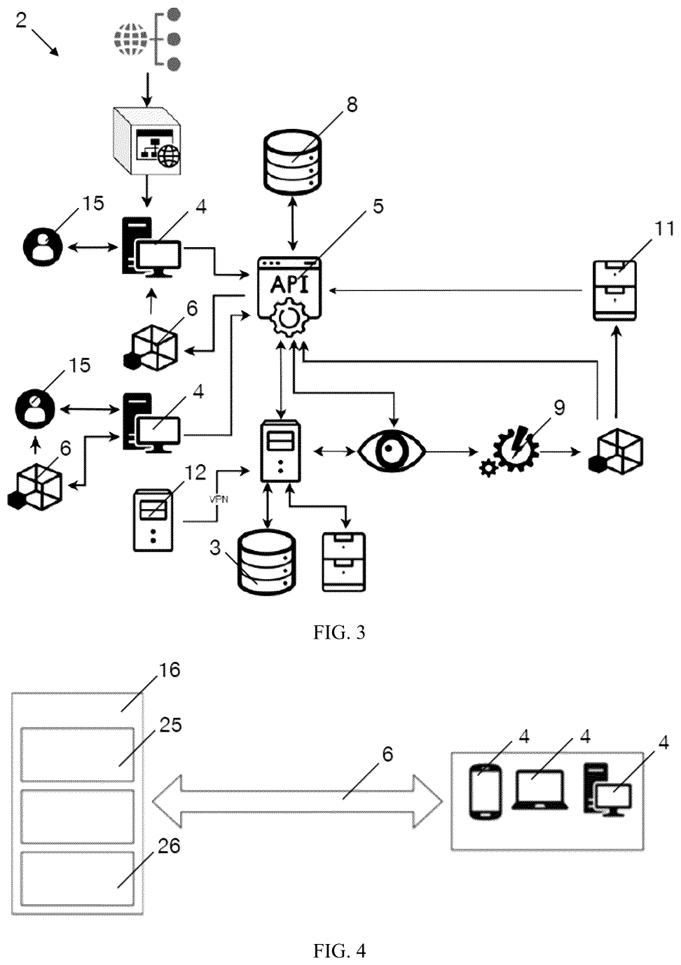

FIGS. 2-3 illustrate two possible arrangements of the data center 2. The data center 2 preferably includes a cloud storage 7, a user database 8, a DICOM converter 9 and a web interface 10. An authorised user 15 can access the system via a displaying means 4 and/or web interface 10. The authorised user 15 interacts with the user database 8 via the API 5. The 3D medical volume storage where the plain 3D medical volumes 25 are stored, is preferably in the cloud storage 7.

FIG. 4 depicts a part of a possible embodiment of the system, showing a board 16, a rendering device 6 and multiple displaying means 4. It is the rendering device's 6 task to render the board 16 for viewing on the displaying means 4. The system may include any number of boards 16 that can be viewed by any number of authorised users 15 on any type of displaying means 4, such as computer, browser, tablet or cellphone, even at the same time. A board 16 may include any number of 3D medical volumes 25 with annotations and/or comments 26 that have been previously saved on the 3D medical volumes 25 by the same or different authorised users 15. A board 16 corresponds to a medical case and to a medical council. The authorised users 15 who added these annotations and/or comments 26 are possibly medical professionals, such as physicians or doctors, who are discussing the medical case. The system allows them working remotely, at the same or at a different time. The rendering device 6 may be a remote server or a local rendering device 6 on the displaying means 4. The boards 16 can have events added to it, such as consultation, surgery, board meeting etc. The authorised users 15 can add a calendar to their calendar service (google calendar, outlook, etc.) via a link. The events of the calendar may contain a link that immediately opens the board 16 or surgery guidance.

The medical collaboration system illustrated in FIGS. 1-4 preferably works as follows. An imaging center 1 obtains at least one, but any number of 2D medical records from a Picture Archiving and Communication System (PACS) server 12 and/or from a disk 13 and/or from an imaging machine 14. The PACS server 12, the disk 13 and the imaging machine 14 are not part of the invention. The imaging center 1 can then send the 2D medical record(s) to a Digital Imaging and Communications in Medicine (DICOM) storage 3. Until this point, the 2D medical record is not modified, changed or edited in any way. The DICOM storage 3 then preferably sends the 2D medical record to a DICOM converter 9 and the DICOM converter 9 removes confidential metadata, such as patient information from the 2D medical record, converting the 2D medical record into a 3D medical volume 25. Each 3D medical volume 25 is preferably provided with a unique identification. The 3D medical volumes 25 can be then sent to a 3D medical volume storage 11 for storing. Then, a user may request access to the medical collaboration system via the API 5. A data center 2 authorises the user by checking a user database 8 in the data center 2; after authorisation, allows the authorised user 15 access. A rendering device 6 can render at least one 3D medical volume on at least one displaying means 4 in response to an input from the authorised user 15. The input can be text or voice or any other way. The at least one displaying means 4 can display the at least one 3D medical volume. The authorised users 15 may create one or more boards 16, each board 16 including at least one 3D medical volume 25. Every board 16 will include the 3D medical volume 25 that are relevant to the medical case or issue. The authorised user 15 can set display parameters and add annotations and/or comments 26 on the at least one 3D medical volume 25 in the board 16. The user database 8 will preferably save and store the board 16 with the annotations and/or comments 26, with the 3D coordinates of the annotations and/or comments 26, and the associated display parameters. Then, the rendering device 6 can render the same board 16 with the saved annotations and/or comments 26 and the associated display parameters on the at least one displaying means 4 in response to an input from the same or a different authorised user 15. This helps understand the medical case better for everyone involved, since this makes it possible for the same or other authorised users 15 to check the added annotations and/or comments 26 in the same settings, from the same angle, etc.

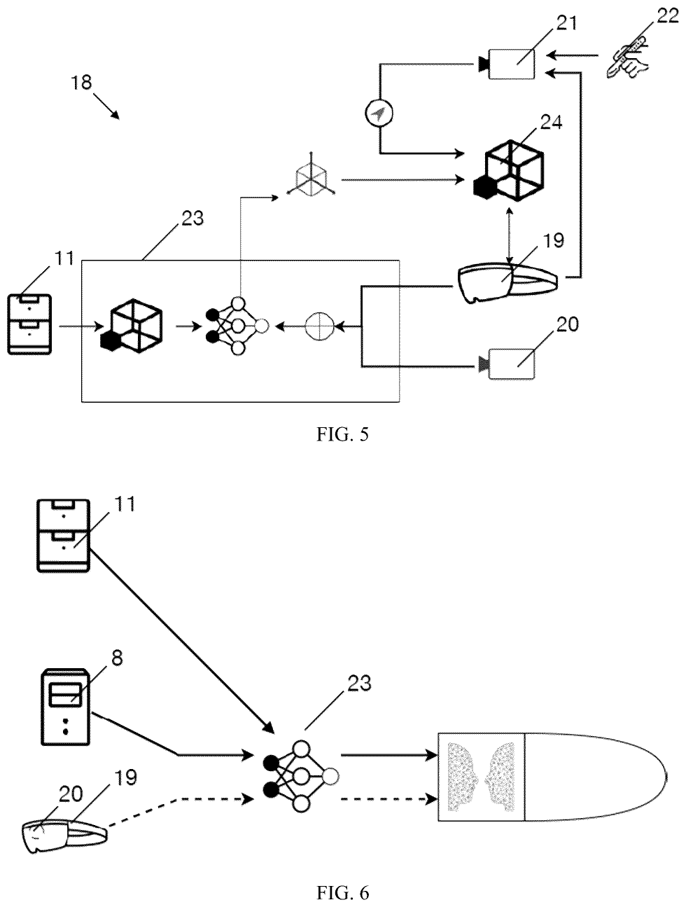

FIG. 5 depicts the optional navigation arrangement 18. The medical collaboration system may include this navigation arrangement 18 for intra-operative use in order to provide real-time optical navigation and visualization for a surgeon during a surgery. Preferably, the navigation arrangement 18 is connected to the data center 2 and includes an XR (Extended Reality) device 19, a depth-camera 20, a tracking sensor 21, registration device 23 and a navigation rendering device 24. The XR device 19 may be a head-mounted XR display or AR glasses and at least one depth-camera 20 might be integrated in the XR device 19. However, the XR device 19 may include multiple depth-cameras 20 as well. The navigation arrangement 18 may further include a data storage server for storing pre-surgically acquired data. The tracking sensor 21 is preferably connected to a surgical tool 22; the surgical tool 22 is not part of the invention. The registration device 23 can be connected to the depth-camera 20 and to the 3D medical volume storage 11. The navigation rendering device 24 can be connected to the user database 8, to the XR device 19, to the tracking sensor 21 and to the registration device 23. These connections can be wired or wireless. The registration device's 23 task is to prepare a virtual image by registering at least one 3D medical volume 25 onto a patient's anatomical structure; and the navigation rendering device's 24 task is to render the virtual image received from the registration device 23 with the saved annotations and/or comments 26 received from the user database 8 on the XR device 19 in real time. During the intra operative scenarios, when a doctor can be wearing an XR device 19, local rendering and offline use is preferred.

When using the embodiments that can be used for intra-operative use, the method for the application of the system may further include the steps of optical positioning and visualization preferably with a single XR device 19. The XR device 19 preferably means AR glasses, worn by the surgeon as a headset. This XR device 19 can show and guide the surgeon such that it projects the annotations and/or comments 26 onto the patient's body (parts) in real-time in order to assist the surgeon, make the surgeries safer and quicker. The XR device 19 can also show the required route of the surgical tools 22 to guide the surgeon even better. These steps are all done without the use of physical markers. Therefore, surgery preparations can be a lot shorter and less risky. The steps included in this method are preferably as follows. An authorised user 15 chooses a board 16, i.e. a medical case, a surgeon—who is also an authorised user 15—preferably wears the XR device 19 as a headset. At least one depth-camera 20, which is a separate element or is integrated in the XR device 19, sends a 3D point cloud of a patient's anatomical structure to a registration device 23; and the 3D medical volume storage 11 sends a 3D point cloud of the 3D medical volume 25 to the same registration device 23. The registration device 23 then does the calculation and registers the two 3D point clouds onto each other. By doing the calculation, i.e. registration, the registration device 23 creates a virtual image that can be displayed by the XR device 19 and shown to the surgeon. The rendering itself is preferably done by a navigation rendering device 24.

The calculation preferably includes the steps of:
pre-sampling vertices of the two 3D point clouds according to the Poisson distribution,
calculating the normal vectors at each point,
sampling a number of sub point clouds from the 3D point clouds,
using a neural net to generate descriptive feature vectors,
comparing these vectors by computing their Euclidean Distance and finding their best matching sub point clouds in the 3D point clouds, coming from the depth-camera 20,
finding the most exactly matching sub point clouds, and using their corresponding Transformation Matrix on the two 3D point clouds.

The 3D point cloud coming from the 3D medical volume storage 11 is preferably registered onto the 3D point cloud coming from the depth-camera 20. If so, the number of sub point clouds sampled from the two 3D point clouds coming from the depth-camera 20 is lower than the number of sub point clouds sampled from the 3D point cloud coming from the 3D medical volume storage 11.

FIG. 6 depicts the registration in a simplified illustration. The registration device 23 in the illustrated embodiment is connected—wired or wireless—to the 3D medical volume storage 11, the user database 8 and the depth-camera 20. In this embodiment, the depth-camera 20 is integrated in the XR device 19. The registration device 23 may also be connected to the DICOM storage 3 in order to be able to receive pre-operative images and/or data.

After the registration is done, the method may further include the steps of the registration device 23 sending the virtual image to a navigation rendering device 24; the user database 8 sending the saved annotations and/or comments 26 from the chosen board 16 to the navigation rendering device 24; and the navigation rendering device 24 rendering the virtual image with the saved annotations and/or comments 26 on the XR device 19 in real time.

The method may further include a precomputation step before the depth-camera 20 sends a 3D point cloud of a patient's anatomical structure to a registration device 23. This precomputation step preferably includes the steps as follows:
pre-sampling vertices of the 3D point cloud coming from the 3D medical volume storage 11 according to the Poisson distribution,
calculating the normal vectors at each point,
sampling a number of sub point clouds from the 3D point cloud coming from the 3D medical volume storage 11,
using a neural net to generate descriptive feature vectors.

As the above description shows, the optical registration and visualization during the intra-operative step is handled completely without the use of physical markers, making the system quicker, safer, more efficient and less expensive than existing registration methods. Another important feature of the system is that it does not diagnose the patients or give any automatic diagnosis in any steps. The diagnosis is done by the medical experts.

Other variations than those described above can be understood and effected by a person skilled in the art. In the claims, the word “including” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. The reference signs used in the claims shall not be construed as limiting the scope. Unless otherwise indicated, the drawings are intended to be read (e.g., cross-hatching, arrangement of parts, proportion, degree, etc.) together with the specification, and are to be considered a portion of the entire written description of this disclosure. As used in the description, the terms “horizontal”, “vertical”, “left”, “right”, “up” and “down”, simply refer to the orientation of the illustrated structure as the particular drawing figure faces the reader.

What is claimed is:

1. A medical collaboration system for a pre-operative collaborative assessment, comprising
an imaging center, a data center, a Digital Imaging and Communications in Medicine (DICOM) storage, at least one displaying means, an application programming interface (API) and a rendering device;
the data center comprising a cloud storage, a user database, a DICOM converter, and a web interface;
the cloud storage comprising a 3D medical volume storage;
the imaging center being connected to the data center and the imaging center being configured to obtain 2D medical records from a Picture Archiving and Communication System (PACS) server and/or from a disk and/or from an imaging machine and send the 2D medical records to the DICOM storage;
the API being connected to the data center, the at least one displaying means, the rendering device, and the 3D medical volume storage;
the DICOM storage being connected to the DICOM converter and the DICOM storage being configured to send the 2D medical records to the DICOM converter;
the DICOM converter being configured to remove confidential metadata from the 2D medical records, convert the 2D medical records into 3D medical volumes, and send the 3D medical volumes to the 3D medical volume storage for storing;
the user database comprising a list of authorised users;
the rendering device being configured to render at least one 3D medical volume on the at least one displaying means in response to an input from an authorised user;
the at least one displaying means being configured to display the at least one 3D medical volume,
the API being configured to allow the authorised user to create a board, and the board comprising the at least one 3D medical volume;
the API being configured to allow the authorised user to set display parameters and make annotations and/or comments on the at least one 3D medical volume in the board,
the user database being configured to save and store the board with the annotations and/or the comments and associated display parameters; and
the rendering device being configured to render the same board with the annotations and/or the comments and the associated display parameters on the at least one displaying means in response to an input from the same authorised user or a different authorised user.

2. The medical collaboration system according to claim 1, further comprising:
a navigation arrangement for intra-operative use,
the navigation arrangement being connected to the data center and comprising an Extended Reality (XR) device, a depth-camera, a tracking sensor, a registration device and a navigation rendering device;
the tracking sensor being connected to a surgical tool;
the registration device being connected to the depth-camera and to the 3D medical volume storage;
the navigation rendering device being connected to the user database, to the XR device, to the tracking sensor, and to the registration device;
the registration device being configured to prepare a virtual image by registering the at least one 3D medical volume onto an anatomical structure of a patient; and
the navigation rendering device being configured to render the virtual image received from the registration device with the annotations and/or the comments received from the user database on the XR device in real time.

3. The medical collaboration system according to claim 2, wherein the XR device is a head-mounted XR display and at least one depth-camera is integrated in the XR device.

4. The medical collaboration system according to any of claim 1, wherein the rendering device is a remote rendering server.

5. The medical collaboration system according to any of claim 1, wherein the displaying means is one of a cell phone, a tablet, a computer, and a web browser.

6. The medical collaboration system according to claim 1, wherein the DICOM storage is in the imaging center and/or in the cloud storage.

7. A method for applying a medical collaboration system, the method comprising steps of:
an imaging center obtaining a 2D medical record from a PACS server and/or from a disk and/or from an imaging machine,
the imaging center sending the 2D medical record to a DICOM storage;
the DICOM storage sending the 2D medical record to a DICOM converter;
the DICOM converter removing confidential metadata from the 2D medical record, converting the 2D medical record into a 3D medical volume, providing the 3D medical volume with a unique identification and sending the 3D medical volume to a 3D medical volume storage for storing;
a user requesting access to the medical collaboration system via an API;
a data center authorising the user by checking a user database in the data center; after an authorisation, allowing an authorised user access;
a rendering device rendering at least one 3D medical volume on at least one displaying means in response to an input from the authorised user;
the at least one displaying means displaying the at least one 3D medical volume, the authorised user creating a board, and the board comprising at least one 3D medical volume;
the authorised user setting display parameters and making annotations and/or comments on the at least one 3D medical volume in the board,
the user database saving and storing the board with the annotations and/or the comments, with 3D coordinates of the annotations and/or the comments, and associated display parameters; and
the rendering device rendering the same board with the annotations and/or the comments and the associated display parameters on the at least one displaying means in response to an input from the same authorised user or a different authorised user.

8. The method according to claim 7, further comprising steps of
the authorised user choosing the board comprising at least one 3D medical volume;
a depth-camera sending a 3D point cloud of an anatomical structure of a patient to a registration device;
the 3D medical volume storage sending a 3D point cloud of the 3D medical volume to the registration device;
the registration device registering the two 3D point clouds onto each other and creating a virtual image by doing a calculation comprising steps of:
pre-sampling vertices of the two 3D point clouds according to a poisson distribution,
calculating normal vectors at each point,
sampling a number of sub point clouds from the 3D point clouds,
using a neural net to generate descriptive feature vectors,
comparing the descriptive feature vectors by computing an euclidean distance of the descriptive feature vectors and finding best matching sub point clouds of the descriptive feature vectors in the 3D point clouds, coming from the depth-camera,
finding most exactly matching sub point clouds, and using transformation matrixes corresponding to the most exactly matching sub point clouds on the two 3D point clouds.

9. The method according to claim 8, wherein the 3D point cloud coming from the 3D medical volume storage is registered onto the 3D point cloud coming from the depth-camera,
and wherein the number of sub point clouds sampled from the two 3D point clouds coming from the depth-camera is lower than the number of sub point clouds sampled from the 3D point cloud coming from the 3D medical volume storage.

10. The method according to claim 8, further comprising steps of
the registration device sending the virtual image to a navigation rendering device;
the user database sending the annotations and/or the comments from the board to the navigation rendering device; and
the navigation rendering device rendering the virtual image with the annotations and/or the comments on an XR device in real time.

11. The method according to claim 8, further comprising a precomputation step before the depth-camera sends a 3D point cloud of the anatomical structure of the patient to the registration device,
the precomputation step comprising:
pre-sampling the vertices of the 3D point cloud coming from the 3D medical volume storage according to the poisson distribution,
calculating the normal vectors at each point,
sampling a number of sub point clouds from the 3D point cloud coming from the 3D medical volume storage,
using the neural net to generate the descriptive feature vectors.

12. The method according to claim 7, wherein a new board is created for every medical case.

13. The medical collaboration system according to claim 2, wherein the rendering device is a remote rendering server.

14. The medical collaboration system according to claim 3, wherein the rendering device is a remote rendering server.

15. The medical collaboration system according to claim 2, wherein the at least one displaying means is one of a cell phone, a tablet, a computer, and a web browser.

16. The medical collaboration system according to claim 3, wherein the at least one displaying means is one of a cell phone, a tablet, a computer, and a web browser.

17. The medical collaboration system according to claim 4, wherein the at least one displaying means is one of a cell phone, a tablet, a computer, and a web browser.

18. The medical collaboration system according to claim 2, wherein the DICOM storage is in the imaging center and/or in the cloud storage.

19. The medical collaboration system according to claim 3, wherein the DICOM storage is in the imaging center and/or in the cloud storage.

20. The medical collaboration system according to claim 4, wherein the DICOM storage is in the imaging center and/or in the cloud storage.
